SEQUENCE LISTING (6 sequences)

SEQ ID NO: 1 - DNA, 69 bp, Artificial Sequence
  Synthetic Construct
ggggacaagtttgtacaaaaaagcaggcttcgaaggagatagaaccatggcgacgaagctatcgagctt

SEQ ID NO: 2 - DNA, 54 bp, Artificial Sequence
  Synthetic Construct
ggggaccactttgtacaagaaagctgggttcacgtgcgattaggaacagctctt

SEQ ID NO: 3 - DNA, 3123 bp, Artificial Sequence
  Synthetic Construct
atgacgacgaagctatcgagcttctgtttcttaacccatggattagcaggaatctcctgcgagagagaacatggaagttctcgtcgatttttctatctcccttctcgtcgattagtttctacttcgtgtaagatgcgacaacaacgtggtttcgactctagtaaaagacaagaggtcaagaaaggctcccctaaaccgatcctatctataaattcaggtcttcagagcaacaatgatgaggaatctgatctggagaatggttctgcggacagtgtcccgagtcttaagtcagatgctgaaaagggtagtagtattcatggcagcatagatatgaatcatgctgatgagaatcttgaaaagaaagatgatatacaaacaactgaagttactcggcgcaagagtaaaactgcaaagaagaaaggggagagtattcatgctacgattgatattggacatgatgatgggaagaatttagataatattactgtgcctgaggttgcaaaagctttgtcccttaacaaaagtgaaggcgagcagatttcagatggacagtttggggaactaatgacaatgataagaagtgcagaaaaaaatattcttcggcttgatgaagcacgggccactgctcttgacgaccttaacaagattcttagtgataaggaagcgttgcagggagaaatcaatgtcttggaaatgaaattgtctgagactgatgaaaggattaaaactgctgctcaagagaaagcacatgtagagcttctggaagaacagttagagaagcttcgtcatgaaatgatctcccctatagaaagtgatggttatgttctagctcttagcaaagagcttgaaacattgaagctggagaatctatctttgagaaacgatatagaaatgcttaagtcagaacttgacagtgttaaagataccggtgaacgtgtggttgtgttggaaaaggagtgctccggtttggaatcttctgtaaaggatttggaatctaagttatcggtttctcaggaagatgtctcgcagctttctactcttaaaatcgaatgcactgatctatgggcgaaggtagagactctacagctgttgttagatagagctaccaaacaagcagagcaagcagttatagtgttacaacagaaccaagatctaagaaataaggtcgacaaaattgaggaatcacttaaagaagccaacgtttataaagaatcttcagagaaaattcaacagtacaatgagctaatgcaacataaggtgacattacttgaggagcggctcgaaaagtctgatgcagagatattctcatatgttcagttatatcaagaatcgataaaagaattccaggaaacacttgaaagtttgaaggaagaaagcaagaaaaaatcaagagatgaaccggttgatgatatgccttgggattattggagtcggttacttttaactgttgatggatggctgcttgaaaagaaaatagcaagcaacgatgctgacttactgagagacatggtatggaagaaagatcgaagaattcatgatacctatattgacgtcaaagataagaatgaacgtgatgccatctctgcatttctcaagcttgtctcatctccaacaagttcaggattgtatgtcgttcacattgcagctgagatggcacctgtagctaaggtgggaggtttgggagatgttgtggcaggtcttggtaaggcattgcaaagaaaaggtcatctggtggagattattcttcccaaatatgactgtatgcagtatgatcgcgtgcgtgacttaagggctttggatactgttgtggagtcatattttgatgggaagttatataaaaacaaaatctggattggcactgttgaaggtttacctgtacatttcattgaacctcaacatccaagcaaattcttctggagaggacagttttatggagagcaagatgatttcagacgcttctcgtattttagccgagctgcactagagttgcttcttcagtccggcaaaaaacccgacatcatacattgtcatgactggcaaacagcttttgttgcgccgctgtattgggatctgtatgctccaaagggattagattctgcaagaatatgctttacatgtcataattttgagtatcaaggtaccgcgtctgcttcagaattgggatcttgcgggcttgatgttaaccagttaaatagaccagacagaatgcaggatcactcgtctggagatagagtcaatcctgttaagggcgctataattttctcaaacattgtaacaaccgtgtcccctacttatgcacaagaagttcgaacagctgagggaggaaaaggactccattcaacactcaattttcactccaagaaattcattggaatcctcaacggcattgacacagattcatggaatcctgccaccgaccccttcctcaaggctcagttcaacgctaaagatctacaagggaaagaagaaaacaaacacgcccttagaaagcagcttggactttcttcagcggagtcaagacggcctttggttggttgcataacaagattagtaccacagaaaggagttcatctaatcagacatgccatatacagaacattagagttaggtggacaatttgtacttcttggttctagcccggttccacatattcagagggaatttgaaggtattgaacaacagtttaaaagccatgatcatgtccggttgttactgaagtacgatgaagctctgtctcatacgatttacgcagcctccgatttgttcatcattccgtcaatttttgagccttgtggacttacacagatgatcgctatgagatatggctccattccaattgcgcgaaaaactggaggcttaaatgacagtgtcttcgacattgatgatgatacaataccaacacagtttcaaaatggatttacatttcaaaccgcggacgaacagggtttcaattatgcgttggagcgagcatttaatcactacaagaaagatgaagagaaatggatgagacttgtagagaaagtgatgagtatagatttcagctggggatcatcagctacacaatacgaagaactctacacgagatcagtgtccagagcaagagctgttcctaatcgcacgtga

SEQ ID NO: 4 - protein, 1040 aa, Artificial Sequence
  Synthetic Construct
MTTKLSSFCFLTHGLAGISCEREHGSSRRFFYLPSRRLVSTSCKMRQQRGFDSSKRQEVKKGSPKPILSINSGLQSNNDEESDLENGSADSVPSLKSDAEKGSSIHGSIDMNHADENLEKKDDIQTTEVTRRKSKTAKKKGESIHATIDIGHDDGKNLDNITVPEVAKALSLNKSEGEQISDGQFGELMTMIRSAEKNILRLDEARATALDDLNKILSDKEALQGEINVLEMKLSETDERIKTAAQEKAHVELLEEQLEKLRHEMISPIESDGYVLALSKELETLKLENLSLRNDIEMLKSELDSVKDTGERVVVLEKECSGLESSVKDLESKLSVSQEDVSQLSTLKIECTDLWAKVETLQLLLDRATKQAEQAVIVLQQNQDLRNKVDKIEESLKEANVYKESSEKIQQYNELMQHKVTLLEERLEKSDAEIFSYVQLYQESIKEFQETLESLKEESKKKSRDEPVDDMPWDYWSRLLLTVDGWLLEKKIASNDADLLRDMVWKKDRRIHDTYIDVKDKNERDAISAFLKLVSSPTSSGLYVVHIAAEMAPVAKVGGLGDVVAGLGKALQRKGHLVEIILPKYDCMQYDRVRDLRALDTVVESYFDGKLYKNKIWIGTVEGLPVHFIEPQHPSKFFWRGQFYGEQDDFRRFSYFSRAALELLLQSGKKPDIIHCHDWQTAFVAPLYWDLYAPKGLDSARICFTCHNFEYQGTASASELGSCGLDVNQLNRPDRMQDHSSGDRVNPVKGAIIFSNIVTTVSPTYAQEVRTAEGGKGLHSTLNFHSKKFIGILNGIDTDSWNPATDPFLKAQFNAKDLQGKEENKHALRKQLGLSSAESRRPLVGCITRLVPQKGVHLIRHAIYRTLELGGQFVLLGSSPVPHIQREFEGIEQQFKSHDHVRLLLKYDEALSHTIYAASDLFIIPSIFEPCGLTQMIAMRYGSIPIARKTGGLNDSVFDIDDDTIPTQFQNGFTFQTADEQGFNYALERAFNHYKKDEEKWMRLVEKVMSIDFSWGSSATQYEELYTRSVSRARAVPNRT

SEQ ID NO: 5 - DNA, 25 bp, Artificial Sequence
  Synthetic Construct
catatggagaactgatgaaaggatt

SEQ ID NO: 6 - DNA, 18 bp, Artificial Sequence
  Synthetic Construct
ctcgagtctttataaacg

(19) United States
(12) Patent Application Publication
(10) Pub. No.: US 2012/0102597 A1
(43) Pub. Date: Apr. 26, 2012

(54) METHOD FOR THE PRODUCTION OF TRANSGENIC PLANTS HAVING HIGH STARCH AND BIOMASS CONTENT AND YIELD

(75) Inventors: Francisco José Muñoz Pérez, Pamplona (ES); Jun Li, Pamplona (ES); Javier Pozueta-Romero, Pamplona (ES); Sandy Raynaud, Pamplona (ES); Paula Ragel De La Torre, Pamplona (ES); Ángel Mérida Berlanga, Pamplona (ES); Miren Edurne Baroja, Pamplona (ES); Manuel Montero, Pamplona (ES)

(73) Assignee: IDEN BIOTECHNOLOGY, Pamplona (ES)

(21) Appl. No.: 13/258,656

(22) PCT Filed: Mar. 17, 2010
(86) PCT No.: PCT/ES2010/070158

§ 371 Date: Dec. 9, 2011

(30) Foreign Application Priority Data
Mar. 24, 2009 (ES) P200930009

(51) Int. Cl. A01H 5/00; C12N 1/21; C12N 15/63; A01H 1/06; C12N 5/10

(52) U.S. Cl. 800/284; 435/419; 435/414; 435/412; 435/417; 435/320.1; 435/252.2; 800/298; 800/317.2; 800/317.3; 800/320; 800/320.2; 800/320.1

(57) ABSTRACT

Process for the production of transgenic plants that have high content and yield of starch and biomass. The starch synthases (SSs) in plants (including SSIV) and glycogen synthase (GlgA) in bacteria catalyse the transfer of the glucosidic part of the ADP-Glucose molecule (the activated donor of glucose) to a pre-existing α(1, 4)-glucan. However, in contrast to the other SSs, SSIV is able to add glucose units to maltotriose. Also, in contrast to other soluble SSs, SSIV is bound to the starch granule. This invention describes for the first time how to obtain plants that have high levels and yields of starch and biomass as a consequence of the expression of genes coding for SSIV.

### FIELD OF THE INVENTION

The present invention is in the fields of genetic engineering and plant physiology. Specifically, the invention comprises a process for the production of transgenic plants with a high content and yield of starch and biomass; it also includes the vectors used for transforming plant cells, the transformed plant cells themselves, the transgenic plants obtained by this process and their uses.

### STATE OF THE ART

The main ways of storing reserve carbohydrates are glycogen (in animals and bacteria) and starch (in plants). In plants, the starch accumulates in large quantities in organs such as seeds and tubers and is a fundamental constituent of the human diet. Starch is also a renewable and fully biodegradable material, often used in the paper, cosmetics, pharmaceutical and food industries, as well as being used as a fundamental component for the manufacture of biodegradable plastics, paints with low environmental impact and bioethanol.

The biosynthesis of starch is a complex process requiring the concerted action of various enzyme activities such as sucrose synthase, phosphoglucomutase, ADP-glucose pyrophosphorylase and various types of glucosyltransferases, commonly named starch synthases (SS) and starch ramification and deramification enzymes (1).

SS in plants and glycogen synthase (GIgA) in bacteria catalyse the transfer of the glucosidic half of the ADP-Glucose molecule (the activated donor of glucose) to a pre-existing α(1, 4)-glucan. The same SSs have been found in all photosynthetic organisms that accumulate starch and are denominated: SSI, SSII, SSIII, SSIV and GBSSI. This high degree of conservation indicates that each of these proteins performs different functions in the process of creation of a starch granule (2). Thus, for example, GBSSI is involved in the production of amylose, while SSI, SSII and SSIII are involved in the production of short starch chains and medium and long amylopectin chains respectively (3).

SSIV is the least known of the protein family known as soluble SSs. Its amino acid sequence is between 30% and 50% different from that of SSI, SSII and SSIII (4, 5). Despite its name, its SS activity has still not been demonstrated. Furthermore, the idea has recently arisen that SSIV does not cover the field of action and the function of the other SSs (6). However, there is evidence to suggest that SSIV can be involved in the determination of the number of starch granules in the plastid (7).

There are numerous references showing that the reduction in the activity of SSI, SSII and SSIII brings about a reduction in starch levels and a change in the structure and composition of the granule (8, 9). Mutants of Arabidopsis without SSIV accumulate reduced levels of starch because, although the amylose/amylopectin balance and molecular structure of the amylopectin are normal, they only produce one granule of starch per chloroplast (7).

In contrast to what was hoped, transgenic plants over-expressing GIgA of Escherichia coli accumulate low starch content (10). Although ectopic expression of SSI, SSII and SSIII has been used as a strategy to increase starch content (WO 00/66745) and modify the properties of starch such as the phosphate content (WO2007/009823) (11-13) and the amylose/amylopectin balance (WO 2006/084336; WO 2002/018606), there is no experimental evidence to indicate that SSIV has SS activity or that the ectopic expression of SSIV can be used as a biotechnological strategy to increase starch content, yield and biomass accumulation in plants. In the present invention, after demonstrating that SSIV is a SS with properties that are totally different to those of the soluble SSs (SSI, SSII, SSIII), we describe for the first time that over-expression of SSIV is a biotechnological strategy for the production of transgenic plants with high levels of starch and high yields of starch and biomass.

### DESCRIPTION OF THE INVENTION

### Brief Description of the Invention

The present invention refers to a process for the production of transgenic plants with a high content and yield of starch and biomass by the ectopic expression of SSIV. The present invention also refers to the transgenic plants characterised by these properties.

The technical effects shown in the present invention can be extrapolated to any type of plant organ such as tubers, leaves, fruit and seeds as well as to any type of plant such as, for example: Arabidopsis, potato, tobacco, tomato, rice, barley, wheat and corn. The results shown in the present invention were achieved for AtSSIV, the gene coding for SSIV in A. thaliana, expressed both constitutively under the control of the 35S promoter and also under the control of a specific tuber promoter (the potato gene promoter). It should be noted that constitutive expression was particularly preferred. The results shown in the present invention were achieved after over-expressing any SSIV isoform and sequence (the particularly preferred from was that of Arabidopsis SSIV). That is, any promoter that is expressed in plants and produces over-expression of either AtSSIV or any other isoform of SSIV are encompassed by the present invention.

For the purpose of the present invention, the following terms are defined:
Transgenic plant: plant where the genome has been modified by genetic engineering with the aim of obtaining different and/or improved biological properties compared to the wild control plant (WT) when both are cultivated under the same conditions.
Transformed plant cell: are plant cells with a genetic alteration resulting from the introduction and expression of genetic material that is external to its genome.
Over-expression of SSIV: a plant over-expresses the SSIV enzyme when the intensity of the band obtained in a Western Blot of a transformed plant extract is significantly higher than that of an extract of a WT plant cultivated in the same conditions and at the same time.
High starch content: as used in the present invention, this expression directly refers to a statistically significant value that is at least 10% higher than values found in control plants.
High biomass productivity: as used in the present invention, this expression directly refers to a statistically significant increase, which is defined as the increase in fresh weight of transgenic plants during their development that is faster than that of wild plants.
SSIV activity: The activity of the SSIV enzyme consists in transferring units of glucose from ADP-Glucose to maltotriose and polyglucans such as starch, amylose, amylopectin and glycogen.

### BRIEF DESCRIPTION OF THE FIGURES

FIG. 1. Restriction map of the pAtSSIV plasmid resulting from the cloning of a complete cDNA coding for the Arabidopsis thaliana AtSSIV gene in the pGEM-T easy (Promega) vector.

FIG. 2. (a-h) Comparison of the amino acid sequences of AtSSI, AtSSII, AtSSIII and AtSSIV. The amino acids that are conserved in all the SSs are highlighted in black. The fragment of AtSSIV used to obtain specific antibodies against this protein is indicated with a bold black line.

FIG. 3. Restriction map of the pGEX-4T3_FragSSIV plasmid used for the synthesis of the peptide necessary for the production of the specific antibody against SSIV.

FIG. 4. Stages of the construction of the pK2GW7,0-AtSSIV binary plasmid (alternatively designated pKan-35S-AtSSIV) necessary for the transformation of plants with Agrobacterium tumefaciens.

FIG. 5. Restriction map of the pET-AtSSIV plasmid necessary for expression of mature SSIV in E. coli.

FIG. 6. Stages in the construction of the pAtSSIV-GFP binary plasmid necessary for the transformation of plants with Agrobacterium tumefaciens.

FIG. 7. Zymogram of SS activity using glycogen as substrate. The SSIV enzyme is separated by electrophoresis in a gel containing glycogen. To provide the signal shown in the figure, the gel was incubated in a solution with ADP-Glucose and later in Lugol's solution, giving rise to the dark band shown. The staining is due to the affinity of Lugol's solution for long chain glucose polymers.

FIG. 8. Specificity of the substrate. In vitro assay of SSI, SSII, SSIII and SSIV with different malto-oligosaccharides as substrates.

FIG. 9. SSIV is capable of complementing the “glycogen-less” phenotype of AgIgAP cells. Template of iodine staining after 12, 24 and 36 hours of incubation of (A) AgIgAP, (B) AgIgAP expressing GIgA and (C, D) AgIgAP expressing SSIV. Figure D shows an amplification of the iodine stain pattern after 36 hours incubation of AgIgAP cells expressing SSIV.

FIG. 10. Subcellular localisation of AtSSIV. The illustration shows the fluorescence produced in Arabidopsis plant cells transformed with AtSSIV-GFP that have been subjected to analysis by a D-Eclipse C1 (NIKON) confocal microscope equipped with an Ar 488 excitation laser, a BA515/30 filter for green emission, a BA650LP filter for red emission and a light detector. In the photographs it can been seen (arrows) that SSIV-GFP is located on the surface of starch grains. chlor: chlorophyll; GFP: “Green Fluorescent Protein”, is the fluorescent protein fused to SSIV capable of emitting fluorescence that is viewed in the confocal microscope.

FIG. 11. Analysis by Western Blot (A) and quantification (B) of the levels of SSIV protein in the wild ecotype Col-0 (WT) and in the transgenic lines that over-expressed the AtSSIV gene (L10, L11, L12 and L13) after integrating the 35S-AtSSIV construction into their genome making use of the A. tumefaciens DSM 19675 strain. In (C), analysis by Western Blot of SSIV in potato tubers expressing AtSSIV after integrating the 35S-AtSSIV construction into their genome making use of the A. tumefaciens DSM 19675 strain. Transgenic plants are labelled 2, 6, 7, 8 and 9.

FIG. 12. Levels of starch in Arabidopsis plant leaves cultivated in greenhouse conditions with a cycle of 16 hours light/8 hours darkness. The white circles correspond to wild Arabidopsis thaliana plants, ecotype Col-0. The black circles correspond to transgenic Arabidopsis plants over-expressing the AtSSIV gene coding for Arabidopsis thaliana SSIV.

FIG. 13. Starch content in tubers of wild potato plants and potato plants expressing AtSSIV after integrating the 35S-AtSSIV construction into their genome making use of the A. tumefaciens DSM 19675 strain, cultivated in greenhouse conditions. The wild tubers analysed are labelled as WT. The transgenic plants are labelled 2, 6, 7, 8 and 9. The values shown correspond to the average and standard deviation of 10 different plants per line.

FIG. 14. Starch content in tubers of wild potato plants and potato plants expressing AtSSIV after integrating the 35S-AtSSIV construction into their genome making use of the A. tumefaciens DSM 19675 strain, cultivated in field conditions. The wild tubers analysed are labelled as WT. The transgenic plants are labelled 7, 8 and 9. The values shown correspond to the average and standard deviation of 30 different plants per line.

FIG. 15. (A) Change in fresh weight of wild Arabidopsis thaliana plants, ecotype Col-0 (white circles) and transgenic Arabidopsis plants over-expressing the AtSSIV gene (black circles), both cultivated in greenhouse conditions throughout their growth phase. The data are the average of three measurements. Each measurement was made by weighing the above-ground parts of 5 plants and dividing the value obtained by five. The bars indicate the standard deviation of the measurements. (B) Visual comparison of Arabidopsis ecotype Col-0 plants (left side of the photograph) and transgenic Arabidopsis plants over-expressing the AtSSIV gene (right side of the photograph).

FIG. 16. Subcellular localisation of AtSSIV in amyloplasts of potato plant tubers over-expressing AtSSIV after integrating the p35S-AtSSIV construction into their genome making use of the A. tumefaciens DSM 19675 strain, cultivated in field conditions. The illustration shows the fluorescence produced in potato plant tubers transformed with AtSSIV-GFP that have been analysed by a D-Eclipse C1 (NIKON) confocal microscope equipped with an Ar 488 excitation laser, a BA515/30 filter for green emission, a BA650LP filter for red emission and a light detector. In the photographs, it can be seen that SSIV-GFP is located at the poles of the starch granules of the tubers of these transgenic plants (indicated by arrows). White bar: 5 μm.

FIG. 17. Southern Blot of transgenic potato plants over-expressing AtSSIV after integrating the p35S-AtSSIV construction into their genome making use of the A. tumefaciens DSM 19675 strain. The figure shows the presence of a single insertion of the p35S-AtSSIV construction in these plants. Non-transformed plants (WT) do not show this construction in their genome. The transgenic plants belong to different lines: 2, 7, 8 and 9.

FIG. 18. Amylose/Amylopectin balance expressed as % amylose in non-transformed control potato plant tubers (WT) and potato plants over-expressing AtSSIV after integrating the p35S-AtSSIV construction into their genome making use of the A. tumefaciens DSM 19675 strain. The data shown in the figure are for plants cultivated in field conditions. The values shown are the average and the standard deviation of the tubers of 30 different plants per line.

FIG. 19. Glucose (A), fructose (B) and sucrose (C) content in non-transformed control potato plant tubers (WT) and in potato plants over-expressing AtSSIV after integrating the p35S-AtSSIV construction into their genome making use of the A. tumefaciens DSM 19675 strain. The values shown correspond to the average and standard deviation of 30 different plants per line, cultivated in field conditions. The concentration of each of the sugars is expressed as μmol/g tuber fresh weigh.

FIG. 20. Protein content in non-transformed control potato plant tubers (WT) and in potato plants over-expressing AtSSIV after integrating the p35S-AtSSIV construction into their genome making use of the A. tumefaciens DSM 19675 strain. The values shown correspond to the average and standard deviation of 30 different plants per line, cultivated in field conditions. The protein concentration is expressed as mg/g tuber fresh weight.

### DETAILED DESCRIPTION OF THE INVENTION

One of the objects described in the present invention refers to the process for obtaining transgenic plants with a high content and yield of starch and biomass, characterised by the transformation of wild plants with an expression vector comprising a nucleotide sequence coding for an enzyme with SSIV activity and the expression of said nucleotide sequence inside the transformed plant.

In a preferred embodiment, the process of the invention is characterised in that the level of SSIV expression inside the transformed plant is at least twice the level of SSIV expression in the wild plant.

In another preferred embodiment, the process of the invention is characterised in that the nucleotide sequence comprising the expression vector used for transforming the wild plant is SEQ ID NO: 3, coding for SEQ ID NO: 4.

In another preferred embodiment, the process of the invention is characterised in that the expression vector used for transforming the plant is Agrobacterium tumefaciens DSM 19675 that comprises the pK2GW7,O. AtSSIV plasmid.

Another object of the present invention refers to the cells transformed with an expression vector, preferably a plasmid, that comprises a nucleotide sequence coding for a protein or protein fragment with SSIV activity.

In a preferred embodiment, the cells of the invention are characterised in that they are transformed with an expression vector selected from: Agrobacterium tumefaciens DSM 19675, plasmid pET-AtSSIV or plasmid pGEX-4T3_FragSSIV, preferably with the Agrobacterium tumefaciens DSM 19675 expression vector. These cells also belong to any of the following plant species: potato (Solanum tuberosum), tobacco (Nicotiana tabacum), barley (Hordeum vulgare), rice (Oryza sativa), corn (Zea mays) or Arabidopsis (Arabidopsis thaliana).

Another object of the present invention refers to the use of said cells for the production of starch and/or biomass.

Another object of the present invention refers to bacterial cells, as described above, and that are characterised in that they have been transformed with a bacterial plasmid selected from: pET-AtSSIV or pGEX-4T3_FragSSIV and belong to an E. coli strain selected from: BL21(DE3), BL21(DE3)AgIgAP or BL21(DE3)AgIgCAP.

Another of the objects of the present invention refers to the use of bacterial cells transformed with the pET-AtSSIV plasmid for the production of an enzyme with SSIV activity, described above.

Another of the objects of the present invention refers to the use of bacterial cells transformed with the pGEX-4T3_FragSSIV plasmid for the production of antibodies against a specific fragment of an enzyme with SSIV activity, described above.

Another object of the present invention refers to the Agrobacterium tumefaciens DSM 19675 expression vector, characterised in that it comprises the pK2GW7,O_AtSSIV plasmid coding for an enzyme with SSIV activity.

Another object of the present invention refers to the pET-AtSSIV plasmid characterised in that it codes for an enzyme with SSIV activity.

Another object of the present invention refers to the pGEX-4T3_FragSSIV plasmid characterised in that it codes for an antigenic fragment of an enzyme with SSIV activity.

Another object of the present invention refers to transgenic plants characterised in that they are transformed with the Agrobacterium tumefaciens DSM 19675 expression vector, characterised in that it comprises the pK2GW7,O_AtSSIV plasmid coding for an enzyme with SSIV activity, and having a high content and yield of starch and biomass in comparison with non-transformed wild plants.

In a preferred embodiment, the transgenic plants of the invention are characterised by showing a level of expression of SSIV that is at least twice that observed in the non-transformed wild plant.

In another preferred embodiment, the transgenic plants of the invention are characterised by showing a starch and/or biomass content that is at least 10% higher than the starch and/or biomass content of wild non-transformed plants, cultivated under the same conditions and at the same period.

In another preferred embodiment, the transgenic plants of the invention are characterised in that they are selected from a group comprising: potato (Solanum tuberosum), tobacco (Nicotiana tabacum), barley (Hordeum vulgare), rice (Oryza sativa), corn (Zea mays) or Arabidopsis (Arabidopsis thaliana).

Another object of the present invention refers to the use of the transgenic plants described above for the production of carbohydrates, selected from: starch, glucose, fructose and sucrose and also for the production of biomass.

Obtaining cDNA Coding for SSIV

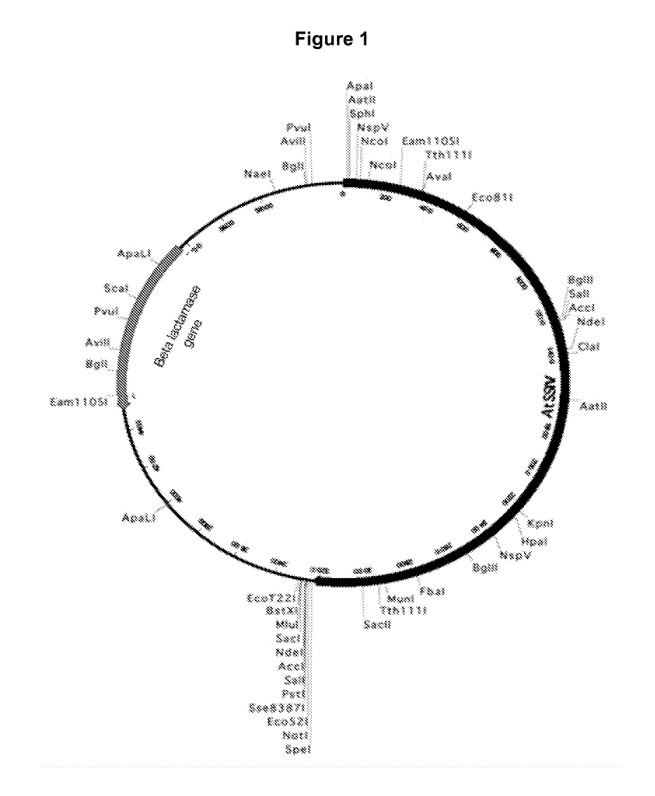

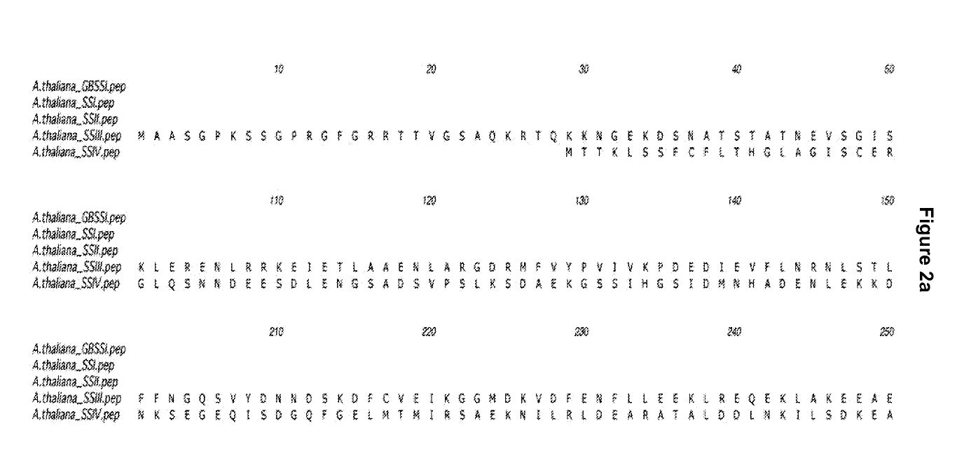

AtSSIV is coded for by the At4g18240 (or AtSSIV) gene. Starting from its nucleotide sequence, specific oligonucleotides were synthesised for the AtSSIV gene. These oligonucleotides were used for RT-PCR amplification of the complete fragment of cDNA coding for AtSSIV, starting from the total RNA of Arabidopsis leaves. The amplified fragment was cloned into the pGEM-T easy (Promega) vector giving rise to the pAtSSIV plasmid (FIG. 1) that was amplified in the XL1-Blue host bacterium.

### Obtaining Specific Polyclonal Antibodies Against the AtSSIV Protein

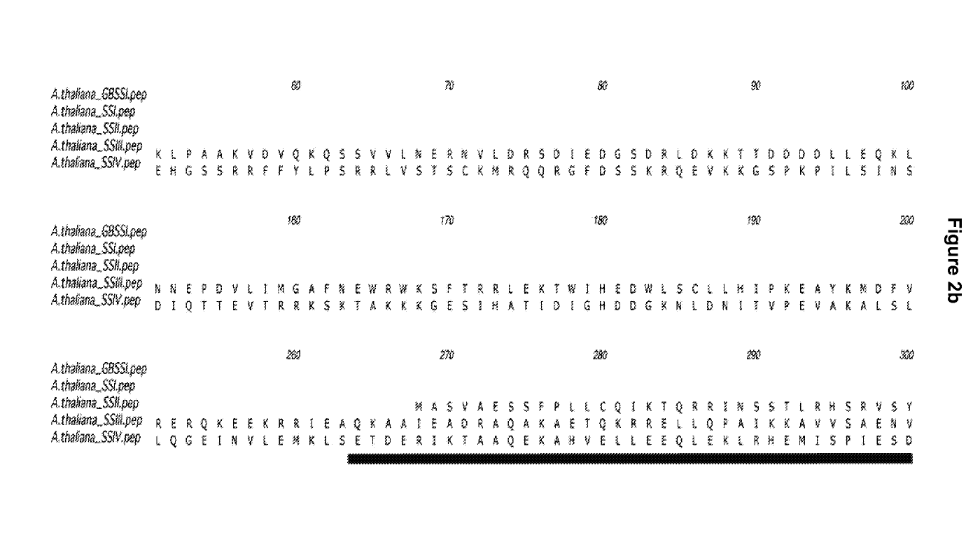

A fragment of the amino terminal region of the protein not showing homology with the other SSs in Arabidopsis (FIG. 2) was selected as the antigenic fragment for obtaining a polyclonal antibody against AtSSIV. Specifically, the region between amino acids Glutamic 236 and Glutamic 414 of the AtSSIV amino acid sequence was used. The oligonucleotides characterised by SEQ ID NO: 5 and 6 were used for cloning the cDNA sequence coding for this fragment.

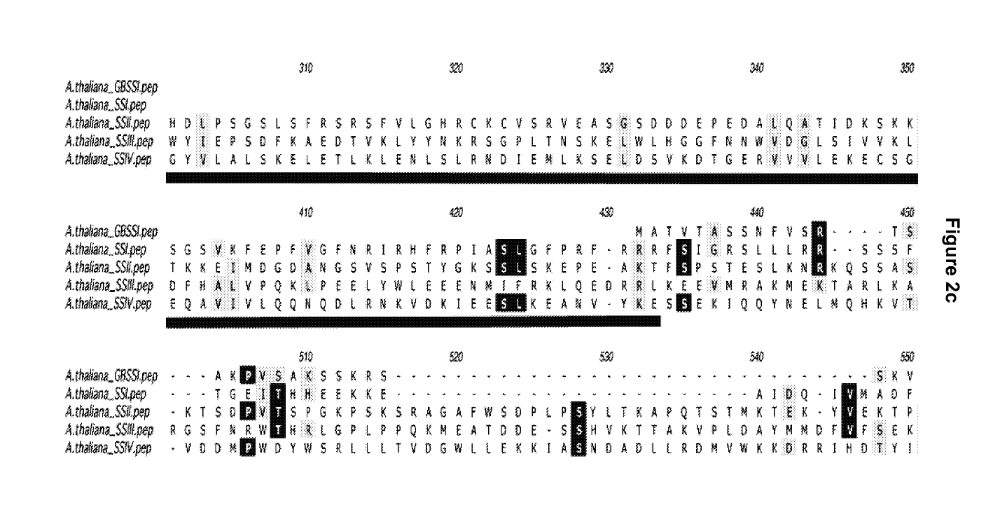

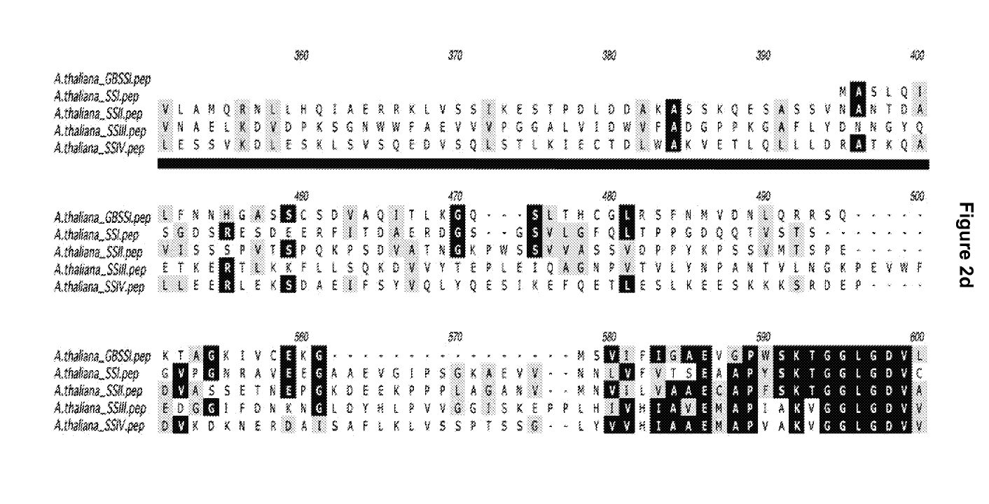

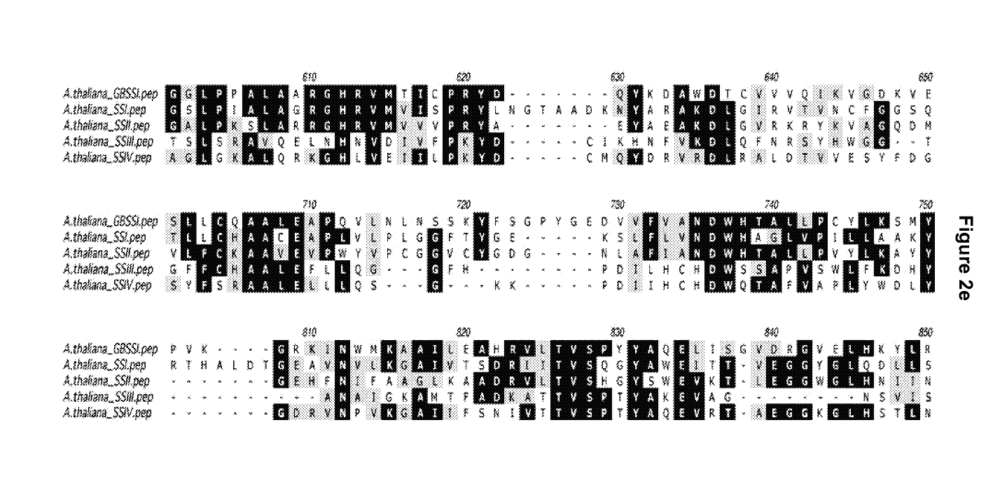

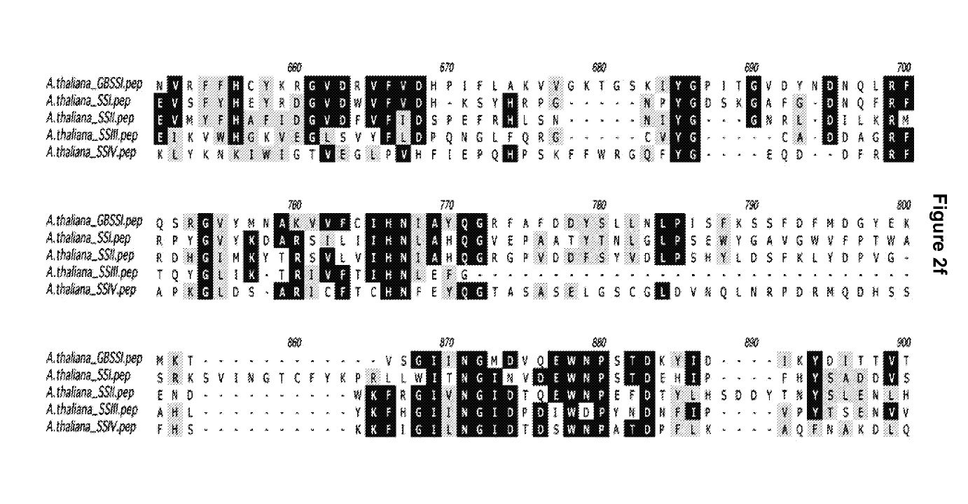

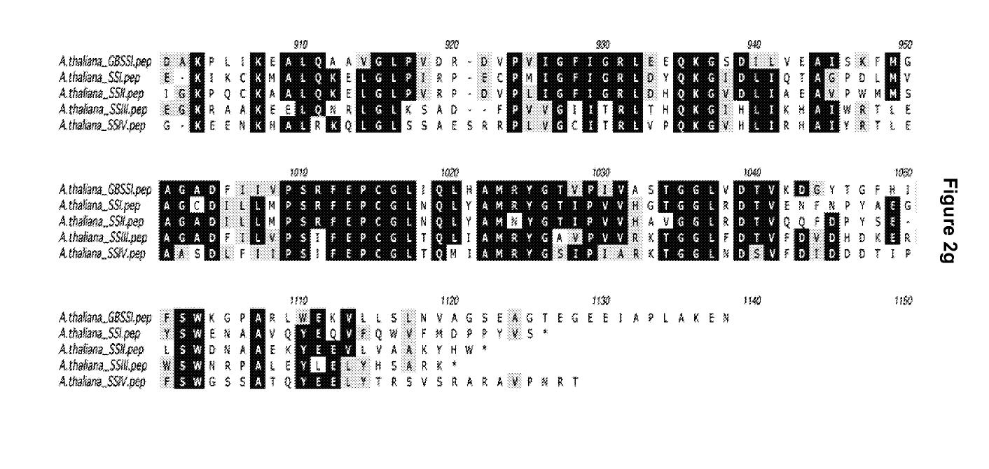

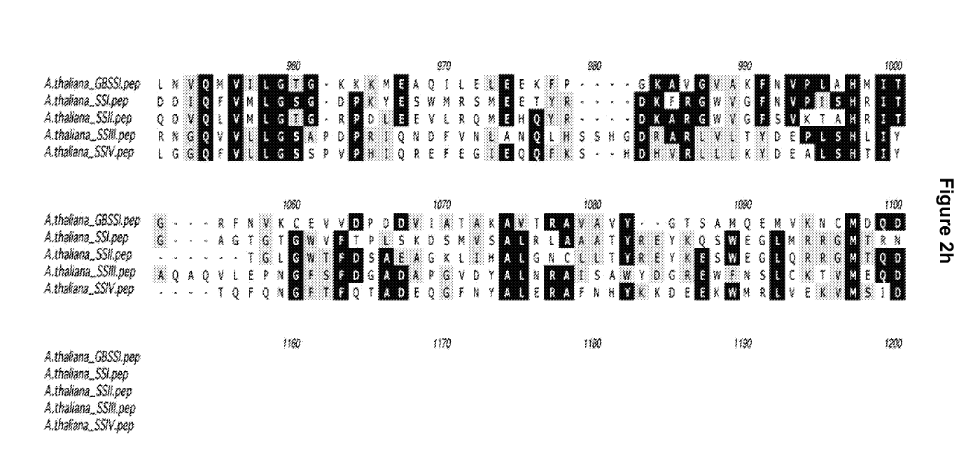

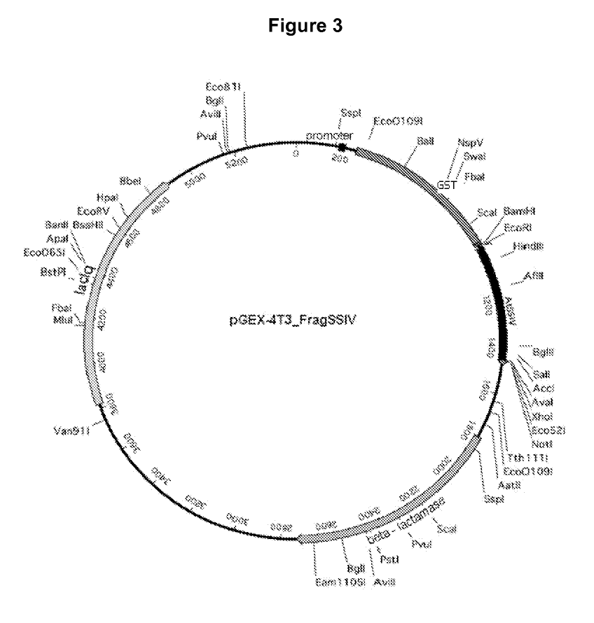

The 512 base-pair fragment was amplified by PCR using these oligonucleotides and cDNA (SEQ ID NO: 3) of the first chain obtained from mRNA of leaves as primers. The oligonucleotides introduce restriction sites for the Ndel and Xhol enzymes at the 5′ and 3′ ends respectively of the amplified fragment. These were used for cloning the cDNA fragment into the pGEX-4T (Amersham Biosciences) expression vector, giving rise to the pGEX-4T3_FragSSIV plasmid (FIG. 3). This expression vector contains the sequence coding for the glutathione S-transferase (GST) protein. The cloning of the cDNA fragment of AtSSIV into the vector was carried out respecting the reading frame marked for the gene coding for GST, allowing translational fusion of the AtSSIV polypeptide fragment with the carboxy-terminal of the GST protein. The construction was confirmed by sequencing the DNA and the strain E. coli BL21 (DE3) was transformed with it.

Then, the expression and purification of the GST-SSIV fusion polypeptide was carried out with Glutathione-Agarose and the subsequent purification of the AtSSIV polypeptide fragment from GST by cleavage with thrombin and binding of the GST to a glutathione matrix. The expression of pGEX-4T3_FragSSIV took place by the addition of 1 mM isopropyl-D-thiogalactopyranoside (IPTG) in 100 ml cell culture when the optical density of the culture was 0.6. After 2 additional hours of culture, the cells were centrifuged at 10,000 g for 5 minutes, resuspended in 50 mM HEPES (pH 7.0) and sonicated. The supernatant containing the recombinant AtSSIV fragment fused with GST (GST-SSIV) was passed through a Glutathione Sepharose (GE Healthcare) affinity column. After washing the column to remove the unbound proteins, the SSIV fragment was eluted by treatment with thrombin, which cleaves the bond of the SSIV fragment with the GST protein, the latter remaining bound to the affinity column. The fragment of purified recombinant AtSSIV was mixed with Freund's complete adjuvant (in a ratio of 50/50) and then distributed into three equal aliquots. They were sent to the Animal Production and Experimentation Centre of Seville University, where rabbit polyclonal antibodies were obtained against this polypeptide. Finally, the anti-SSIV antibody was purified by FPLC using a Protein A Sepharose column (Amersham Bioscience).

Obtaining Transgenic Plants that Over-Express AtSSIV

The constitutive over-expression of AtSSIV required the production of a binary plasmid, the production process of which is illustrated in FIG. 4. AtSSIV was amplified by PCR using pAtSSIV and then cloned into pDONR/Zeo, giving rise to the pDONR/Zeo-AtSSIV plasmid. Using pDONR/Zeo-AtSSIV and pK2GW7,0 (14), the pK2GW7,0-AtSSIV (or pKan-35S-AtSSIV) plasmid was obtained, which has the 35S constitutive promoter, AtSSIV and the 35S terminator. pK2GW7,0-AtSSIV was introduced into A. tumefaciens by electroporation, giving rise to the DSM 19675 strain, which was deposited in the “German National Resource Centre for Biological Material” on 18 September 2007, address: DMSZ, Mascheroder Weg 1 b D-38124 (Braunschweig, Germany). This strain was used to transform potato and Arabidopsis plants following the protocols described in the literature (15, 16).

Obtaining AgIgAP and AgIgCAP Escherichia coli Cells that Over-Express AtSSIV

The sequence of AtSSIV coding for the mature AtSSIV protein was amplified by PCR starting from pAtSSIV and later cloned into pET-45b(+) (Novagen) giving rise to the pET-AtSSIV plasmid as shown in FIG. 5. pET-AtSSIV was introduced by electroporation into BL21(DE3) AgIgAP and AgIgCAP E. coli strains (17). These strains do not have glycogen synthase activity that could interfere with the SS activity. The over-expression of AtSSIV took place by the addition of 1 mM isopropyl-D-thiogalactopyranoside (IPTG) in 100 ml cell culture when the optical density of the culture was 0.6. After 2 additional hours of culture, the cells were centrifuged at 10,000 g for 5 minutes, resuspended in 50 mM HEPES (pH 7.0) and sonicated.

### Identification of SSIV

SSIV is a SS (EC 2.4.1.21) that transfers glucose from ADP-Glucose to the end of a starch or glycogen chain (or other type of polysaccharide consisting of glucose molecules bound to each other by α-(1,4) type covalent bonds) by the creation of a new α-(1,4) type bond. It also has the unusual feature of using maltotriose as substrate. The identification of SSIV can be achieved by any of the following ways: (a) by zymograms, (b) by analysis of the incorporation of radioactivity from radioactively labelled ADP-Glucose into glucose polysaccharides, (c) by complementation of the “glycogen-less” phenotype of the AgIgAP strain of E. coli, (d) by immunoblots making use of specific antibodies against AtSSIV and (e) by confocal microscopy analysis of the subcellular localisation of SSIV fused with the green fluorescence protein (GFP).
By zymograms: SSIV electrophoretically separated on a native gel (50 mM GlyGly/NaOH, pH 9; 100 mM (NH₄)₂SO₄; (5 mM 3-mercaptoetanol; 5 mM MgCl₂, 0.25 g/l BSA) containing glycogen (or any other type of polysaccharide of glucose molecules bound by α-(1,4) bonds between them) and which has been incubated in a solution with ADP-Glucose will give rise to dark bands in Lugol's solution (0.5% I₂/1.5% Kl). The staining is due to the affinity of Lugol's iodine for long chain glucose polymers.
By measurement of the radioactivity of glucose polymers generated from radioactively labelled ADP-Glucose: SSIV incubated as described in (3) with radioactively labelled ADP-Glucose in a solution of 50 mM glycine/NaOH (pH 9.0), 100 mM (NH₄)₂SO₄, (5 mM 3-mercaptoethanol, 5 mM MgCl₂ containing maltotriose (10 mg/ml), 1 mg/ml glycogen or any other type of long polysaccharide of glucose molecules bound together with α-(1,4) bonds) will give rise to a radioactively labelled glucose polymer as a result of the incorporation of the radioactively labelled glucose from ADP-Glucose. The radioactivity incorporated in such a polymer can be measured by using a scintillation counter.
By complementation of the “glycogen-less” phenotype of the AgIgAP strain of E. coli: the AgIgAP insertion mutant of E. coli does not accumulate glycogen as it does not have the glgA gene coding for GIgA. This enzyme is responsible for the synthesis of glycogen from ADP-Glucose found in the cell. Therefore, the identification of SSIV activity in AgIgAP cells of E. coli is manifest by the observation of the accumulation of glycogen in the mutant transformed with pET-AtSSIV.
By Western Blot: in the case of potato plants, AtSSIV is detected by use of the specific anti-AtSSIV antibody by the Western Blot method described in (18). In the case of Arabidopsis, the antigen-antibody complex is detected by incubation with a secondary rabbit anti-IgG conjugated with peroxidase and using the ECL Advanced® detection kit, (Amersham), which gives rise to a chemoluminescent product. The light signal is detected and quantified by a Bio-Rad ChemiDoc image capture system using “Quantity One” image analysis software also from Bio-Rad.
By analysis of its subcellular localisation by confocal microscopy: potato and/or Arabidopsis plants were transformed with the AtSSIV-GFP chimeric construction obtained as illustrated in FIG. 6. The plants were subjected to confocal microscopy observation to identify the subcellular location of the GFP fluorescence.

### Determination of the Soluble Sugar and Starch Content

Leaves and tubers were crushed in a mortar with liquid nitrogen. The starch was quantified by a spectrophotometric method consisting of the total degradation of the starch to glucose residues by the action of the amyloglucosidase enzyme and subsequent quantification of the glucose using an enzyme assay coupled with hexokinase and glucose-6-phosphate dehydrogenase enzymes (7). The amylose/amylopectin balance was determined by a spectrophotometric method (19).

### Deposit of Microorganisms According to the Budapest Treaty

The microorganisms used in the present invention were deposited in the “German National Resource Centre for Biological Material” on 18 September 2007, at DMSZ, Mascheroder Weg 1 b D-38124 (Braunschweig, Germany) with deposit number DSM 19675.

### EXAMPLES OF EMBODIMENT OF THE INVENTION

Examples are given below showing in detail the process for obtaining transgenic Arabidopsis and potato plants with a high starch content, high yield and high biomass productivity as a consequence of the increase in SSIV activity. The modes of embodiment, examples and figures that follow are provided for illustration purposes only and are not limiting of the present invention.

### Example 1

Obtaining Complete cDNA Coding for AtSSIV

Knowledge of the nucleotide sequence of the AtSSIV gene coding for AtSSIV enabled the creation of two specific primers, the sequences of which in the 5′-3′ direction are SEQ ID NO: 1 and SEQ ID NO: 2. Making use of these primers and of RNA from Arabidopsis leaves, a complete cDNA for AtSSIV (At4g18240) was amplified by conventional RT-PCR methods and was cloned into pGEM-T easy (Promega) (FIG. 1).

The nucleotide sequences of the amplified DNA and the amino acid sequence deduced are shown in SEQ ID NO: 3 and SEQ ID NO: 4 respectively.

### Example 2

Identification of the Product with SSIV Activity

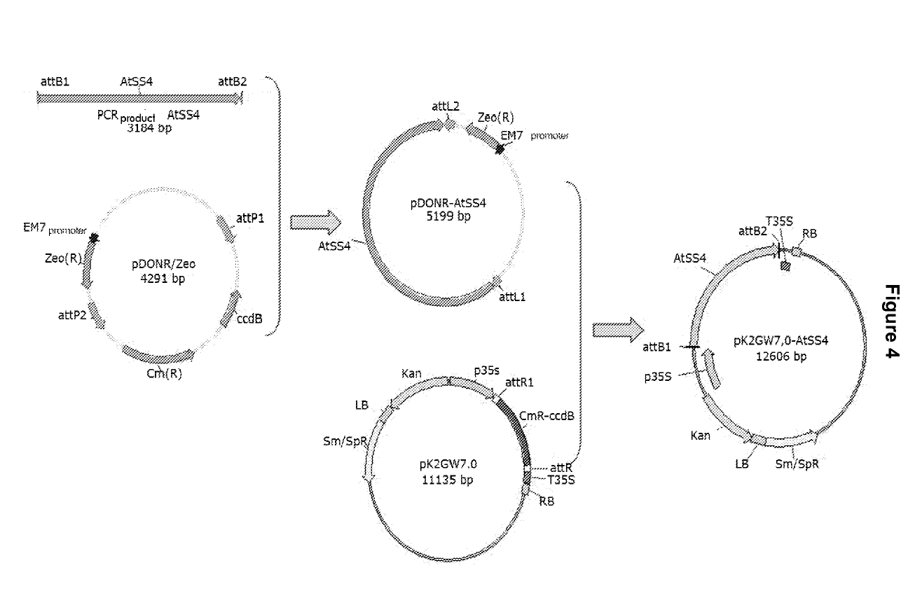

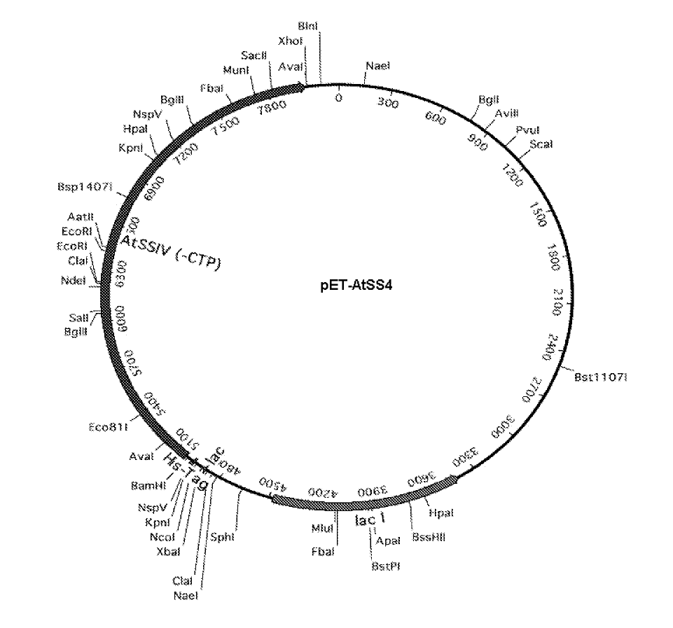

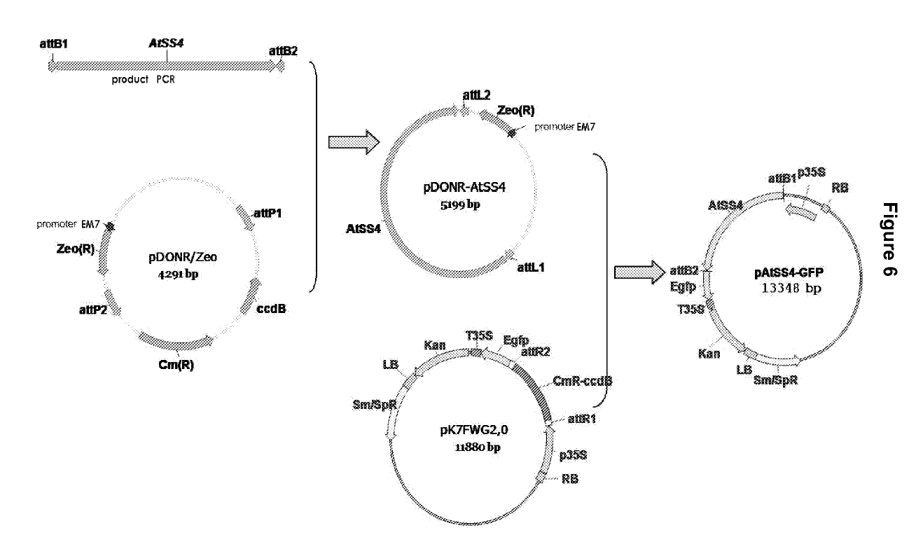

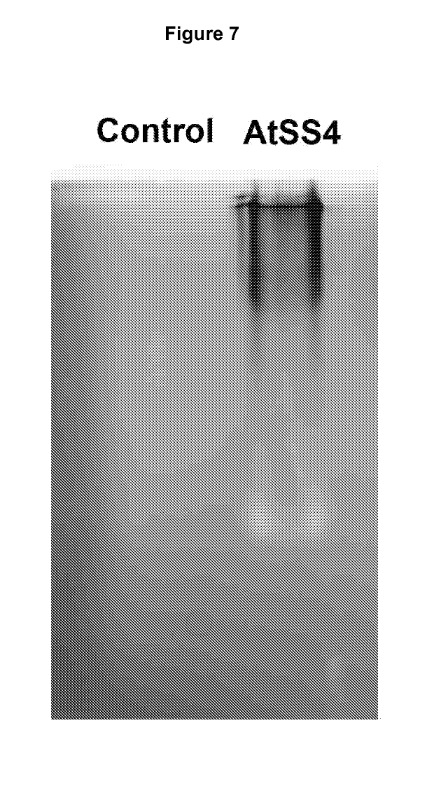

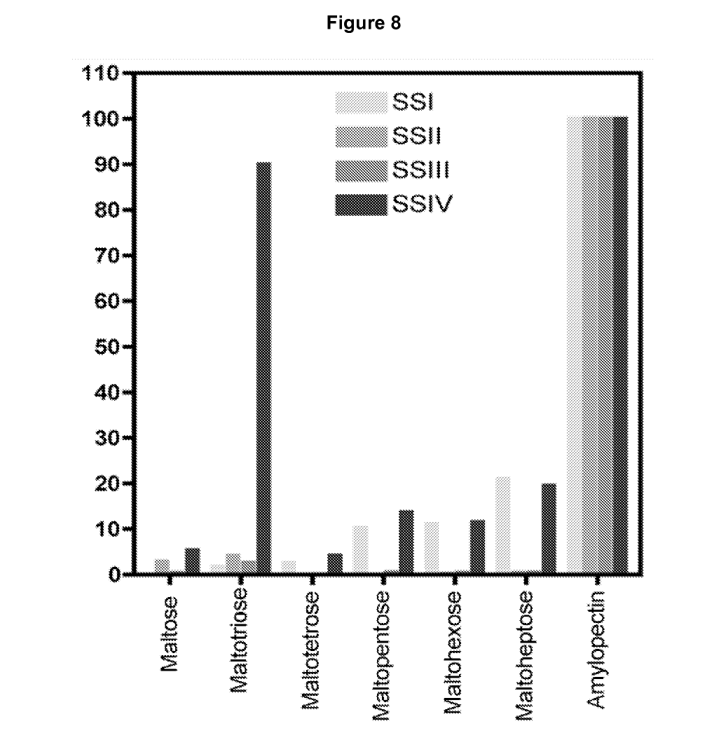

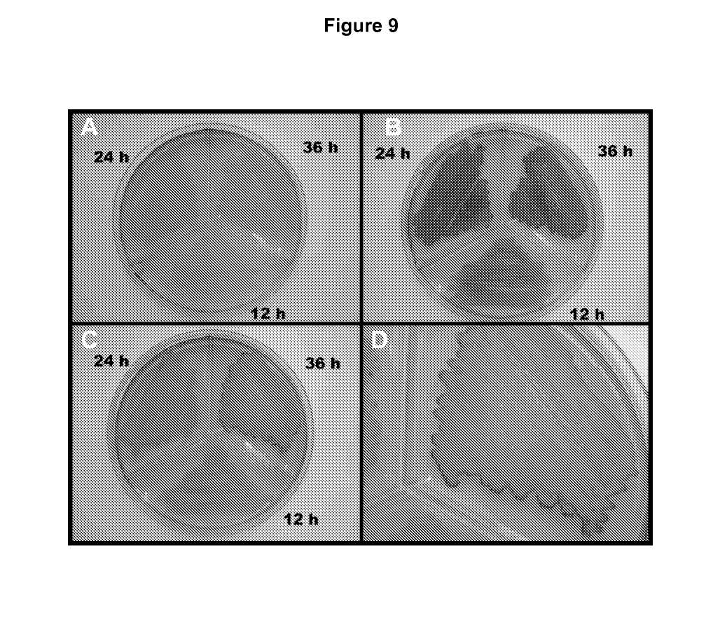

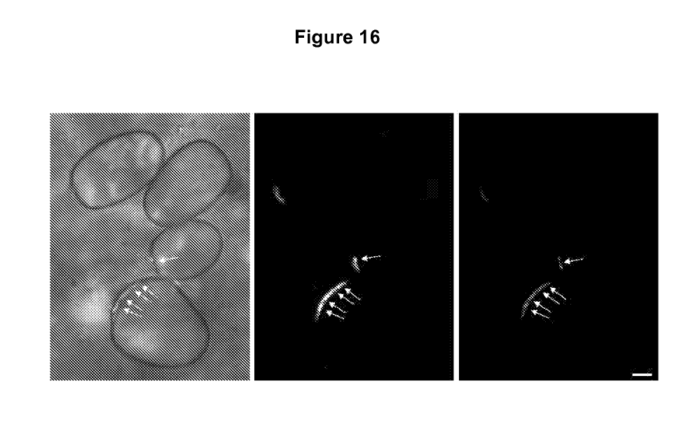

Zymogram identification: 100 μg of protein from crude extracts of E. coli BL21(DE3) AgIgCAP cells transformed with pET-45b(+) or with pET-AtSSIV were subjected to electrophoresis in native conditions on a 7.5% polyacrylamide gel without SDS, which contained 0.3% (p/v) of glycogen from pig liver (SIGMA). After incubating the gel overnight at ambient temperature in 50 mM GlyGly/NaOH pH 9; 100 mM (NH₄)₂SO₄; (5 mM 3-mercaptoethanol; 5 mM MgCl₂, 0.25 g/l BSA and 1 mM ADP-Glucose), it was incubated in an iodine solution (Lugol) composed of 0.5% I₂/1.5% KI. The presence of new glycogen chains was revealed owing to the appearance of a dark band in the gel. This dark band is due to the affinity of Lugol for long chain glucose polymers, so that where a SS had stopped its migration and had elongated polyglucan chains by the addition of glucose residues with α-(1,4) bonds, a dyed and darker area was seen in the gel. As can be seen in the zymogram of FIG. 7, BL21(DE3) AgIgCAP cells transformed with pET-AtSSIV showed a glycogen elongating activity dependent on ADP-Glucose. This activity was absent in BL21(DE3) transformed with pET-45(+).
Identification by incorporation of radioactivity from radioactive ADP-Glucose (FIG. 8). The purified enzymes were incubated at 30 ° C. for 30 min in 100 μl of the following reaction mixture: 50 mM Glygly/NaOH pH 9; 100 mM (NH₄)₂SO₄; 5 mM 3-mercaptoetanol; 5 mM MgCl₂; 0.25 g/l BSA; 1 mM ADP-[U-C] Glucose (3.7 GBq/mol). Lastly, 10 mg/ml of malto-oligosaccharide (with a degree of polymerisation of between 2 and 7) or corn amylopectin were added, depending on the substrate analysed. The reaction was stopped by boiling the sample for 10 min and the glucans produced were elongated by incubation at 30° C. overnight with 7.5 U of rabbit phosphorylase a (Sigma) in the presence of 50 mM Glucose-1-P (final concentration). The reaction was stopped by the addition of 3.5 ml of a solution of 75% methanol and 1% KCl and then centrifuged to precipitate the synthesised glucan. The pellet obtained was washed three times with the same stopping solution and finally the incorporated radioactivity was quantified by the addition of 5 ml of Ready Protein scintillation liquid (Beckman) followed by reading in a scintillation counter model LS 6000 IC (Beckman). The elongation with phosphorylase was omitted when the substrate used was amylopectin. As can be seen in FIG. 8, assays of substrate specificity of SSIV showed that SSIV is capable of transferring glucose molecules from ADP-Glucose to polyglucans such as amylopectin. Malto-oligosaccharides of 4, 5, 6 or 7 glucose units were not good substrates for SSIV. Surprisingly, maltotriose is an excellent substrate for SSIV (as good as amylopectin). This substrate specificity pattern distinguishes SSIV from other SSs because, as can be seen in FIG. 8, SSI, SSII and SSIII do not act efficiently on maltotriose (3).
Identification by complementation of the “glycogen-less” phenotype of the AgIgAP strain of E. coli: as can be seen in FIG. 9A, the AgIgAP cells of E. coli do not accumulate glycogen as they do not have GIgA. The “glycogen-less” phenotype of this strain disappears on ectopically expressing the glgA gene coding for E. coli GIgA (FIG. 9B). In the same way that AgIgAP cells of E. coli transformed with pET-glgA accumulated glycogen, so did the AgIgAP cells of E. coli transformed with pET-AtSSIV (FIG. 9C, 9D).
Identification by subcellular localisation: potato and/or Arabidopsis plants were transformed with the chimeric AtSSIV-GFP construction obtained as illustrated in FIG. 6. The plants were subjected to analysis of the subcellular localisation of GFP fluorescence by D-Eclipse C1 confocal microscope (NIKON) equipped with an Ar 488 excitation laser, a BA515/30 filter for green emission, a BA650LP filter for red emission and a light detector. In the photographs of FIG. 10, in contrast to what occurs with other members of the soluble starch synthase family, SSIV-GFP is bound to starch granules. Equally, the cellular localisation of SSIV in potato plant tubers transformed with the chimeric AtSSIV-GFP construction was analysed. In the photographs of FIG. 16, it can be seen that SSIV-GFP is localised in the poles of the starch granules present in the amyloplasts of the potato plant tubers transformed according to the invention.

These methods of SSIV identification demonstrate that the AtSSIV protein is a SS with glucosyl transferase activity from the donor ADP-Glucose molecule to long chain polyglucan chains such as amylopectin, amylose and glycogen. Also, in contrast to the other SSs, SSIV is able to add glucose units to maltotriose. Finally, SSIV is the only member of the soluble starch synthase family that is associated with the starch granule.

### Example 3

Obtaining and Characterisation of Transgenic Plants that Over-Express SSIV

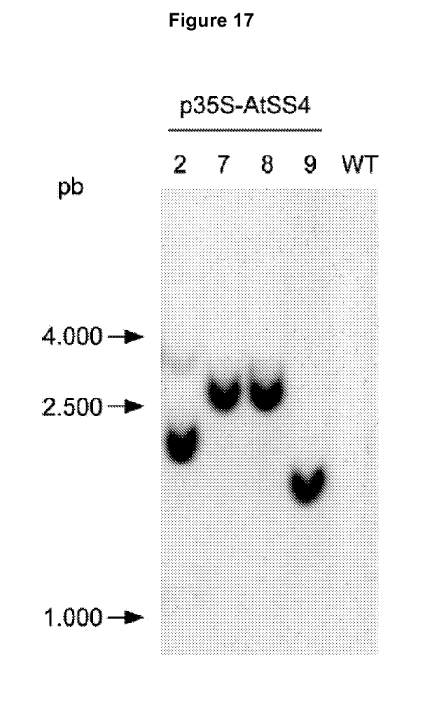

Using the Agrobacterium tumefaciens DSM 19675 strain (that contains the pK2GW7,0-AtSSIV plasmid, alternatively designated as pKan-35S-AtSSIV), transgenic Arabidopsis thaliana and potato (Solanum tuberosum) plants were obtained that over-expressed AtSSIV in a constitutive way. In order to demonstrate that the transgenic plants transformed with the A. tumefaciens DSM 19675 strain included a single insertion of the construction described above, a Southern Blot was performed on these transformed plants. The probe used for detection by this technique was created by radioactively labelling the gene conferring resistance to kanamycin with the isotope dCTP ³²P. As can be seen in FIG. 17, all transgenic potato plant lines (2, 7, 8 and 9) showed a single insertion of the pKan-35S-AtSSIV construction in their genome, while non-transformed control plants did not have this construction in their genome.

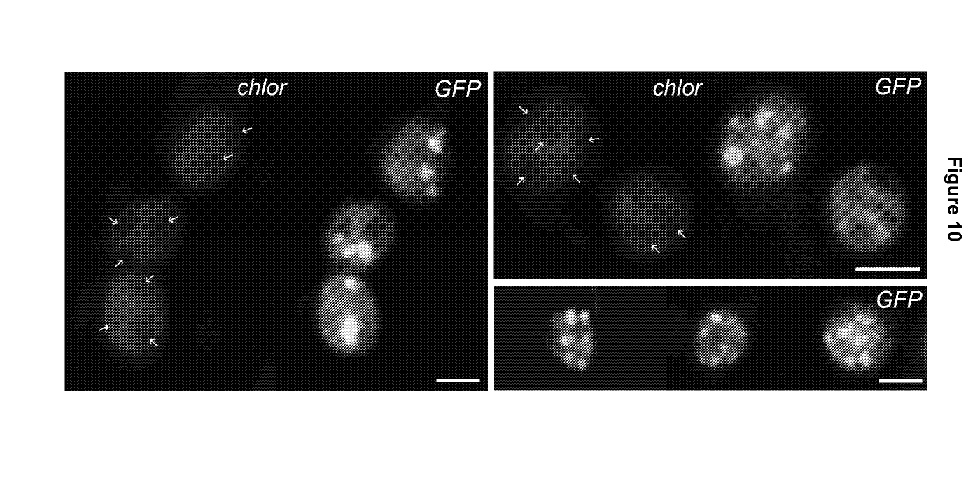

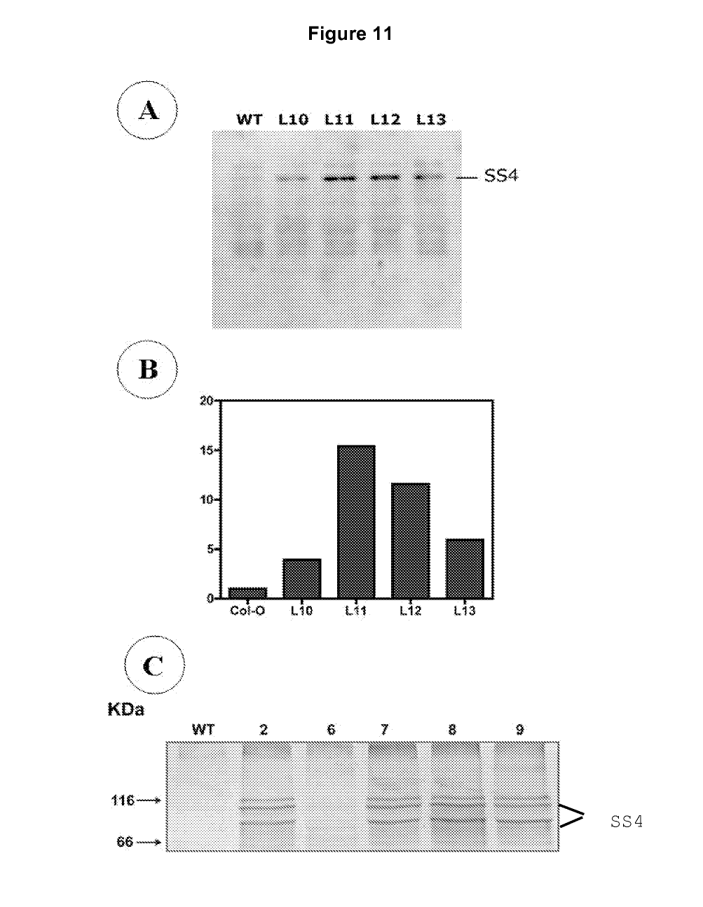

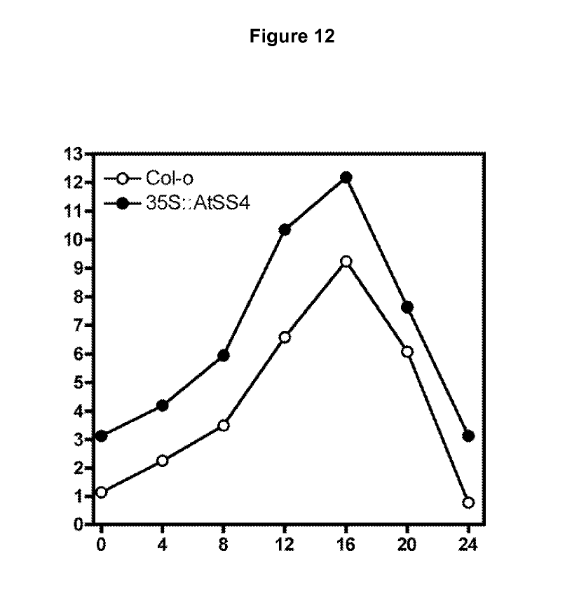

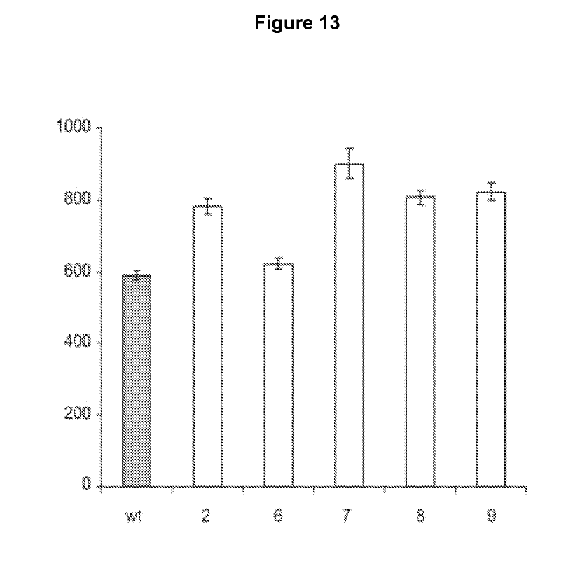

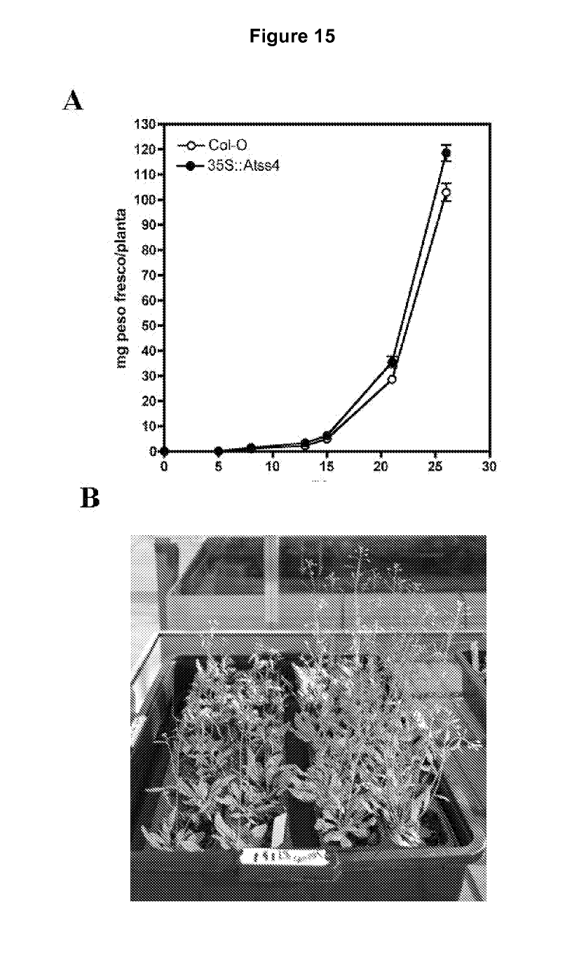

Compared with non-transformed plants, the plants over-expressing AtSSIV accumulated significantly higher levels of a protein of approximately 112 kDa that is recognised by the specific polyclonal antibody against AtSSIV (FIG. 11). In the case of the potato, this protein has internal breaks that give rise to fragments of approximately 80 and 100 kDa. In FIG. 13, it can be seen that the levels of starch in the leaves of Arabidopsis plants over-expressing AtSSIV, cultivated in greenhouse conditions, are significantly higher than those of non-transformed control plants (WT). A positive correlation can also be seen between the levels of expression of SSIV (FIG. 11C) and the levels of starch in tubers of plants grown in greenhouse conditions. In addition, Arabidopsis plants cultivated in greenhouse conditions showed higher yield in the production of biomass and growth to that observed in non-transformed control plants (FIG. 15 A) while their morphology was similar to that of non-transformed plants (FIG. 15 B).

In addition to greenhouse cultivation, transgenic potato plants over-expressing the AtSSIV gene and control plants were cultivated in field conditions. These cultivations were performed between May and September 2009 on a plot of district 25 in Sartaguda (Navarre, Spain). The plants were distributed at random on 50 square meter plots, using 30 plants per line. The separation between rows was 90 cm. The separation between plants on the same row was 35 cm.

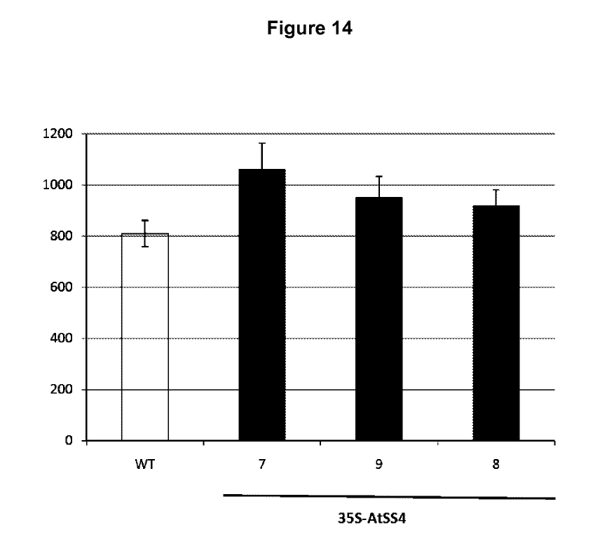

As can be seen in FIG. 14, the potato plant tubers that over-expressed AtSSIV, cultivated in field conditions, accumulated significantly higher levels of starch than the corresponding organs of control plants. These data correlate with the significant increase in the concentration of starch found in the leaves of the transgenic plants of the invention compared to control plants cultivated in greenhouse conditions.

Table 1 shows the starch content of tubers of plants that over-expressed AtSSIV and the tubers of control plants, both of them cultivated in field conditions. The results shown in this table are the average and standard deviation of 30 different plants per line. The significantly different values to those recorded in control plants are indicated in bold. The results shown in Table 1 demonstrate that the tubers of potato plants that over-express AtSSIV showed a significant increase, approximately of 30%, in the concentration of starch expressed as a percentage of dry weight (% DW) compared to the tubers of non-transformed control plants (WT).

The productivity data per unit of area (kg/ha) shown in Table 1 indicate that the tubers of transgenic plants over-expressing AtSSIV showed a significant increase in starch content compared to the tubers of control plants (WT).

Also, the tubers of transgenic plants over-expressing AtSSIV, cultivated in field conditions, produced significantly higher concentrations of starch than the tubers of control potato plants (WT). While the tubers of control plants produced 94.65 g of starch per plant, the plants over-expressing AtSSIV produced between 103.9 and 137 g of starch per plant.

TABLE 1
Quantitative parameters of transgenic plants
over-expressing AtSSIV in field conditions.

| | | 35S-AtSIV | | |
|---|---|---|---|---|
| | WT | SS4-7 | SS4-8 | SS4-9 |
| Tuber starch | 11.3 ± 0.3 | 15.3 ± 0.8 | 13.2 ± 0.3 | 13.7 ± 0.5 |
| (% FW) | | | | |
| Tuber starch | 56.1 ± 9.8 | 80.2 ± 4.5 | 72.5 ± 1.8 | 70.1 ± 2.5 |
| (% DW) | | | | |
| Tuber starch | 94.6 ± 1.2 | 137 ± 2.9 | 98.6 ± 1.4 | 104 ± 2.0 |
| (g/plant) | | | | |
| Tuber starch | 4254 ± 52 | 6091 ± 108 | 4381 ± 160 | 4619 ± 197 |
| (kg/ha) | | | | |

FW: fresh weight;
DW: dry weight.
The significantly different values to those recorded in control plants are indicated in bold.

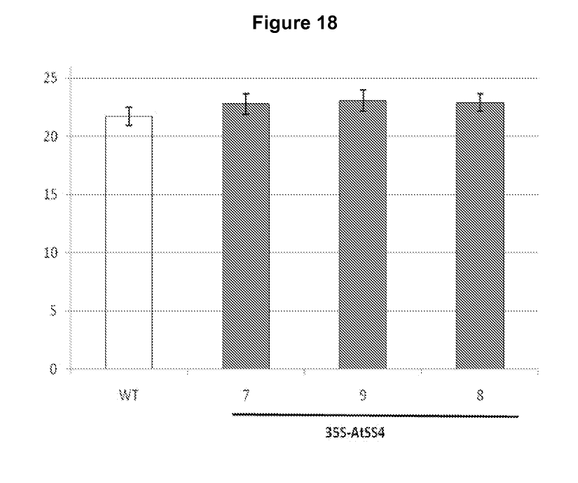

As can be seen in FIG. 18, the amylose/amylopectin balance expressed in percentage of amylose in the tubers of transgenic plants over-expressing AtSSIV was similar to that observed in the tubers of non-transformed control plants. Therefore, although over-expression of AtSSIV brings about an increase in the quantity of starch accumulated in the tubers of potato plants that over-express AtSSIV, the quality of the starch of these transgenic tubers was similar to the quality of the starch in the tubers of non-transformed control plants. That is, the same type of starch is found both in wild non-transformed plants as in the transformed plants of the invention.

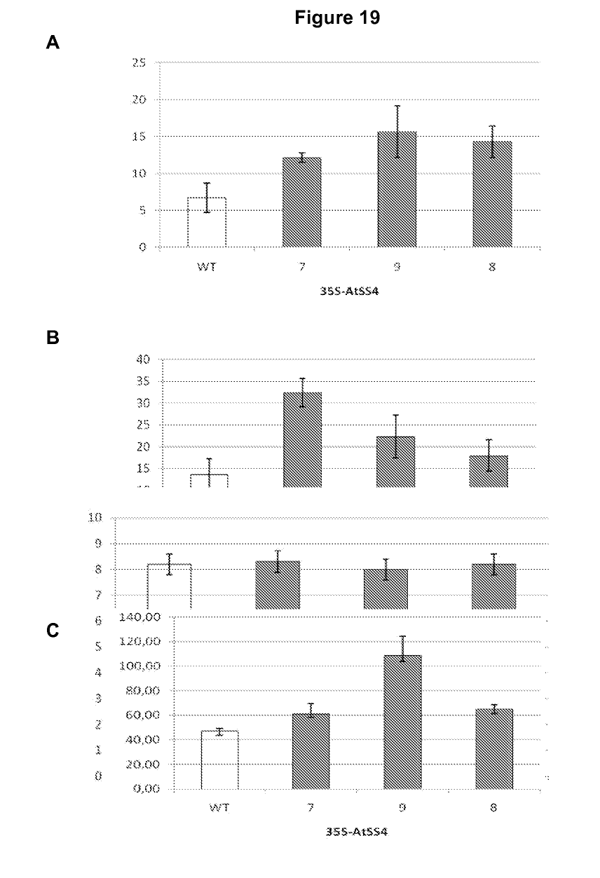

Another of the characteristics defining the transgenic plants of the invention is that they show an increase in soluble sugar content, such as glucose, fructose and sucrose (FIG. 19), in the tubers compared to non-transformed control plants (WT).

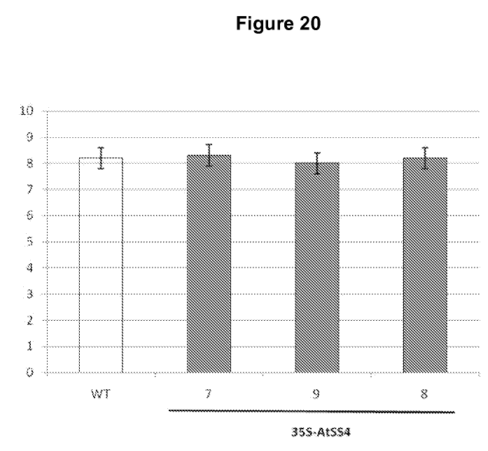

Potato tubers act as reservoirs of starch and proteins. FIG. 20 shows that the protein content of tubers of potato plants over-expressing AtSSIV is similar to that found in the tubers of non-transformed control potato plants. Therefore, this figure demonstrates that the over-expression of AtSSIV in transgenic plants does not alter the protein content of the tubers of these transgenic plants. This demonstrates the specificity of the over-expression of AtSSIV for the specific accumulation of starch in these transgenic plants compared to non-transformed control plants.

### REFERENCES

1. Ball, S., and Morell, M. (2003). From bacterial glycogen to starch: understanding the biogenesis of the plant sttarch granule. Annu. Rev. Plant Biol. 54, 207-233.
2. Ral, J. P., Derelle, E., Ferraz, C., Wattebled, F., Farinas, B., Corellou, F., Buléon, A., Slomianny, M. C., Delvalle, D., d'Hulst, C., Rombauts, S., Moreau, H., Ball, S. (2004). Starch division and partitioning. A mechanism for granule propagation and maintenance in the picophytoplanktonic green alga Ostreococcus tauri. Plant Physiol. 136, 3333-3340.
3. Delvallé, D., Dumez, S., Wattlebled, F., Roldán, I., Planchot, V., Berbezy, P., Colonna, P., Vyas, D., Chatterjee, M., Ball, S., Mérida, A., D'Hulst, C. (2005). Soluble starch synthase I: a major determinant for the synthesis of amylopectin in Arabidopsis thaliana leaves. Plant J. 43, 398-412.
4. Hirose, T., Terao, T. (2004). A comprehensive expression analysis of the starch synthase gene family in rice (Oriza sativa L.). Planta 220, 9-16.
5. Leterrier, M., Holappa, L. D., Broglie, K. E., Beckles, D. . (2008). Cloning, characterization and comparative analysis of a starch synthase IV gene in wheat: functional and evolutionary implications. BMC Plant Biol. Doi: 10.1186/1471-2229-8-98.
6. Zhang, X., Szydlowski, N., Delvallé, D., D'Hulst, C., James, M. G., Myers, A. M. (2008). Overlapping functions of the starch synthases SSII and SSIII in amylopectin biosynthesis in Arabidopsis. BMC Plant Biol. doi:10.1186/1471-2229-8-96.
7. Roldán, I., Wattebled, F., Lucas, M. M., Delvallé, D., Planchot, V., Jiménez, S., Pérez, R., Ball, S., D'Hulst, C., and Mérida, A. (2007). The phenotype of soluble starch synthase IV defective mutants of Arabidopsis thaliana suggests a novel function of elongation enzymes in the control of starch granule formation. The Plant Journal 49:492-504.
8. Edwards, A., Fulton, D. C., Hylton, C. M., Jobling, S. A., Gidley, M., Rossner, U., Martin, C., Smith, A. M. (1999). A combined reduction in activity of starch synthases II and III of potato has novel effects on the starch of tubers. Plant J. 17, 251-261.
9. Jobling, S. A., Westcott, R. J., Tayal, A., Jeffcoat, R., Schwall, G. P. (2002). Production of a freeze-thaw-stable potato starch by antisense inhibition of three starch synthase genes. Nat. Biotechnol 20, 295-299.
10.Shewmaker, C. K., Boyer, C. D., Wiesenborn, D. P., Thompson, D. B., Boersig, M. R., Oakes, J. V., Stalker, D. M. (1994). Expression of Escherichia coli glycogen synthase in the tubers of transgenic potatoes results in a highly branched starch. Plant Physiol. 104, 1159-1166.
11. Lloyd, J. R., Landschütze, V., Kossmann, J. (1999). Simultaneous antisense inhibition of two starch-synthase isoforms in potato tubers leads to accumulation of grossly modified amylopectin. Biochem. J. 338, 515-521.
12. Abel, G. J. W., Springer, F., Willmitzer, L., Kossmann, J. (1996). Cloning and functional analysis of a cDNA encoding a novel 139 kDa starch synthase from potato (Solanum tuberosum L.). Plant J. 10, 981-991.
13.Zhang, X., Myers, A. M., James, M. G. (2005). Mutations affecting starch synthase III in Arabidopsis alter leaf starch structure and increase the rate of starch synthesis. Plant Physiol. 138, 663-674.
14.Karimi, M., Inze, D., Depicker, A. (2002). GATEWAY™ vectors for Agrobacterium-mediated plant transformation. Trends Plant Sci. 7: 193-195.
15. Rocha-Sosa, M., Sonnewald, U., Frommer, W., Stratmann, M., Schell, J., Willmitzer, L. (1989). Both developmental and metabolic signals activate the promoter of a class I patatine gene. EMBO J. 8, 23-29.
16.Clough, S. J., Bent, A. F. (1998). Floral dip: a simplified method for Agrobacterium-mediated transformation of Arabidopsis thaliana. Plant J. 16: 735-43.
17. Morán-Zorzano, M. T., Alonso-Casajús, N., Muñoz, F. J., Viale, A. M., Baroja-Fernández, Eydallin G., Pozueta-Romero, J. (2007). Occurrence of more than one important source of ADPglucose linked to glycogen biosynthesis in Escherichia coli and Salmonella enterica. FEBS Lett. 581, 4423-4429.
18. Towbin, H., Staehelin, T., Gordon, J. (1979). Electrophoretic transfer of proteins from polyacrylamide gels to nitrocellulose sheets: procedures and some applications. Proc. Natl. Acad. Sci. USA 76, 4350-4354.
19.Andersson, M., Melander, M., Pojmark, P., Larsson, H., Bülow, L., Hofvander, P. (2006). Targeted gene suppression by RNA interference: an efficient method for production of high-amylose potato lines” J. Biotechnol. 123, 137-148.

1. Process for obtaining transgenic plants with a high content and yield of starch and biomass, characterised by the transformation of wild plants with an expression vector comprising a nucleotide sequence coding for an enzyme with SSIV activity and the expression of said nucleotide sequence inside the transformed plant.

2. Process according to claim 1, characterised in that the level of SSIV expression inside the transformed plant is at least twice the level of SSIV expression in the wild plant.

3. Process according to claim 1, characterised in that the nucleotide sequence comprising the expression vector used for transforming the wild plant is SEQ ID NO: 3, coding for SEQ ID NO: 4.

4. Process according to claim 1, characterised in that the expression vector used for transforming the plant is Agrobacterium tumefaciens DSM 19675 that comprises the pK2GW7,O_AtSSIV plasmid.

5. Cell transformed with an expression vector, preferably a plasmid, that comprises a nucleotide sequence coding for a protein or a protein fragment with SSIV activity.

6. Cell according to claim 5, transformed with an expression vector selected from: Agrobacterium tumefaciens DSM 19675, the pET-AtSSIV plasmid or the pGEX-4T3_FragSSIV plasmid.

7. Plant cell according to claim 6, characterised in that it has been transformed with Agrobacterium tumefaciens DSM 19675 and belongs to any of the following plant species: potato (Solanum tuberosum), tobacco (Nicotiana tabacum), barley (Hordeum vulgare), rice (Oryza sativa), corn (Zea mays) or Arabidopsis (Arabidopsis thaliana).

8. Bacterial cell according to any of the claim 5 or 6, characterised in that it has been transformed with a bacterial plasmid selected from:
pET-AtSSIV or pGEX-4T3_FragSSIV and belongs to a strain of E. coli selected from: BL21(DE3), BL21(DE3)AgIgAP or BL21(DE3)AgIgCAP.

9. Expression vector Agrobacterium tumefaciens DSM 19675, characterised by comprising the pK2GW7,O_AtSSIV plasmid coding for an enzyme with SSIV activity.

10. pET-AtSSIV plasmid, characterised in that codes for an enzyme with SSIV activity.

11. pGEX-4T3_FragSSIV plasmid, characterised in that codes for an antigenic fragment of an enzyme with SSIV activity.

12. Use of the bacterial cells transformed with the pET-AtSSIV plasmid of claim 8 for the production of an enzyme with SSIV activity.

13. Use of the bacterial cells transformed with the pGEX-4T3_FragSSIV plasmid of claim 8 for the production of antibodies against a specific fragment of an enzyme with SSIV activity.

14. Use of the transformed cell of claim 6 for the production of starch and/or biomass.

15. Transgenic plant, characterised in that it has been transformed with the vector of claim 9 and that has a high content and yield of starch and biomass compared with the non-transformed wild plant.

16. Transgenic plant according to claim 15, characterised in that it shows a level of expression of SSIV of at least twice that observed in the non-transformed wild plant.

17. Transgenic plant according to claim 15, characterised in that it shows a starch and/or biomass content that is at least 10% higher than the starch and/or biomass content of non-transformed wild plants, cultivated under the same conditions and at the same period.

18. Transgenic plant according to claim 15, selected from the group comprising: potato (Solanum tuberosum), tobacco (Nicotiana tabacum), barley (Hordeum vulgare), rice (Oryza sativa), corn (Zea mays) or Arabidopsis (Arabidopsis thaliana).

19. Use of the transgenic plants of claims 15 to 18 for the production of carbohydrates selected from a group including: starch, glucose, fructose and sucrose.

20. Use of the transgenic plants of claims 15 to 18 for the production of biomass.
